(19) United States
(12) Patent Application Publication
(10) Pub. No.: US 2006/0269500 A1
(43) Pub. Date: Nov. 30, 2006

(54) Topical warming composition

(75) Inventors: Jed A. Riemer, Scarsdale, NY (US); Kimberly Burch, Hamilton, NJ (US); Carter B. Green, Stony Point, NY (US)

Correspondence Address: DARBY & DARBY P.C., P. O. BOX 5257, NEW YORK, NY, 10150-5257, US

(73) Assignees: Takasago International Corp. (USA), Rockleigh, NJ (US); Lipo Chemicals Inc., Paterson, NJ (US)

(21) Appl. No.: 11/444,895

(22) Filed: May 31, 2006

Related U.S. Application Data
(60) Provisional application No. 60/595,033, filed on May 31, 2005.

(51) Int. Cl. A61K 8/89; A61K 36/906; A61K 36/67; A61K 31/11

(52) U.S. Cl. 424/70.12; 424/401; 424/756; 424/734; 514/463; 514/699

(57) ABSTRACT

The present invention provides compositions that include (a) a warming agent or a cooling agent and (b) a silicone-containing component. Also provided is a method of increasing the warming effect of a warming agent or the cooling effect of a cooling agent by adding a silicone-containing component to a topical composition containing the warming agent or cooling agent.

### CROSS REFERENCE TO RELATED APPLICATION

This application claims the benefit of priority under 35 U.S.C. § 119(e) of provisional application no. 60/595,033, filed May 31, 2005, which is hereby incorporated by reference in its entirety.

### FIELD OF THE INVENTION

The present application relates to compositions that impart a warming or cooling effect on the user when topically applied.

### BACKGROUND OF THE INVENTION

It is known that substances, including natural isolates, exist which provide a topical warming sensation when applied to skin. Substances which are known to provide this warming effect are referred to as “warming agents” and include capsicum (red pepper powder, tincture, oleoresin, and extract), capsaicin, homo-capsaicin, homo-dihdrocapsaicin, nonanoyl vanillyl amide, nonanoic acid vanillyl ether, vanillyl alcohol alkyl ether derivatives (JP-A-57-9729), isovanillyl alcohol alkyl ether derivatives, ethylvanillyl alcohol alkyl ether derivatives, substituted benzyl alcohol alkyl ether derivatives, 4-(1-menthoxymethyl)-2-(3′-methoxy-4′-hydroxyphenyl)-1,3-dioxolane and analogs (U.S. Pat. Nos. 5,545,424 and 5,753,609), vanillin propylene glycol acetal, ethylvanillin propylene glycol acetal, ginger extract and oil, gingerol, and gingerone. These warming agents may be added as a single ingredient or as part of a fragrance composition to various products to produce a topical warming effect. However, they often cause either skin irritation or exhibit insufficient warming effect and those having good warming effects are of short duration or when used in decreased amounts exhibit insufficient warming and/or duration (See U.S. Pat. Nos. 6,673,844 and 6,570,010). In addition, it is also known that sensate materials, including warming, cooling and tingle, in combination can impart a variety of desirable sensations or effects (See U.S. Pat. Nos. 6,890,597 and 6,780,443).

### SUMMARY OF THE INVENTION

The present invention provides a warming composition that includes (a) at least warming agent; and (b) at least one silicone-containing component. The warming agent may be selected from, for example, vanillyl butyl ether and vanillyl ethyl ether and the silicone-containing component may be selected from, for example, cyclomethicone, cyclopentasiloxane, and dimethicone.The compositions may further comprise cooling agent.

Embodiment of the present invention also provide a cooling composition that includes a cooling agent; and a silicone-containing component.

Also provided is a method of increasing the effect of a sensate material (e.g. a tingling sensate, a cooling sensate or a warming sensate) comprising adding an effective amount of a silicone-containing component to a composition comprising the sensate material.

### DETAILED DESCRIPTION

It has been found that the effect of a sensate material (e.g. a warming agent such as vanillyl alkyl ether) is enhanced when it is combined with a silicone-containing component.

Warming Agents

The warming effect of warming agents (also known as warming sensates) is increased when combined with a compounds that contain silicone (e.g. dimethicone). As used herein warming agents refer to compounds that impart heating or warming sensation upon topical application to the skin, oral cavity, throat or mucous membrane.

In one embodiment of the present invention, the warming agent is selected from compounds represented by the following formula: 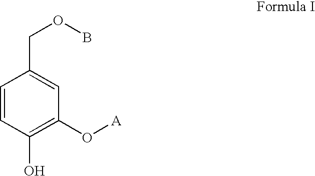
or a dermotologically acceptable salt thereof, wherein A is an unsubstituted, branched or straight-chained C₁-C₃ alkyl group and B is a hydrogen, or an unsubstituted, branched or straight-chained C₁-C₇ alkyl group.

In one embodiment A is C₁ alkyl group. In a preferred embodiment, A is C₁ alkyl and B is a C₂-C₄ alkyl group. In a particularly preferred embodiment, the warming sensate is selected from vanillyl butyl ether (A is a C₁ alkyl group and B is a straight-chained, unsubstituted C₄ alkyl group) and vanillyl ethyl ether (A is a C₁ alkyl group and B is a straight-chained, unsubstituted C₄ alkyl group). Vanillyl butyl ether is commercially available from Takasago, Inc. under the name Hotact® VBE.

In another embodiment the warming sensate is selected from compounds represented by the formula: 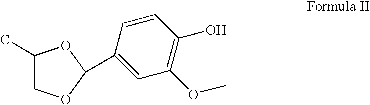
or a dermatologically acceptable salt thereof wherein C is an unsubstituted, branched or straight-chained C₂-C₈ alkyl group, optionally interrupted by an oxygen atom.

In a preferred embodiment C is a unsubstituted, straight-chained C₄-C₅ alkyl group optionally interrupted by an oxygen atom. In a particularly preferred embodiment, warming agents are selected from vanillin-1,2-hexylene glycol acetal (C is an unsubstituted, straight-chained C₄ alkyl group) and vanillin-1-butoxyglycerol acetal (C is an unsubstiuted, straight chained C₅ alkyl group interrupted by an oxygen at the 2 position). Warming agents may also be selected from those disclosed in Japanese patent application No. JP 2005-197205, which is hereby incorporated by reference in its entirety.

Warming agents may also be selected from the group consisting of capsaicin, gingerol, vanillyl ethyl ether, vanillyl propyl ether, vanillyl butyl ether, vanillyl pentyl ether, vanillyl hexyl ether, vanillyl butyl ether acetate, 4-(1-menthoxymethyl)-2-phenyl-1,3-dioxolan, 4-(1-menthoxymethyl)-2-(3′,4′-dihydroxyphenyl)-1,3-dioxolan, 4-(1-menthoxymethyl)-2-(2′-hydroxy-3′-methoxyphenyl)-1,3-dioxolan, 4-(1-menthoxymethyl)-2-(4′-methoxyphenyl)-1,3-dioxolan, 4-(1-menthoxymethyl)-2-(3′,4′-methylenedioxyphenyl)-1,3-dioxolan, 4-(1-menthoxymethyl)-2-(3′-methoxy-4′-hydroxyphenyl)-1,3-dioxolan, red pepper oil, red pepper oleoresin, ginger oleoresin, nonylic acid vanillyl amide, jambu oleoresin, Zanthoxylum piperitum extract, sanshool I, sanshool II, sanshoamide, black pepper extract, chavicine, piperine, spilanthol, or those warming agents disclosed in U.S. Pat. No. 6,780,443, which is hereby incorporated by reference in its entirety.

As would be understood by one of ordinary skill in the art, the same compound may act differently depending on its use level in the composition. For example, a compound may act as a warming agent at a certain use level, yet the same compound will act as a tingling sensate at a higher use level.

Cooling Agents

When cooling agents (also known as cooling sensates) are added to compositions containing warming agents, the cooling agent increases the warming effect of the warming agent. See, e.g. U.S. Pat. No. 6,780,443 which is hereby incorporated by reference. Accordingly, topical warming compositions of the present invention may contain a “cooling agent” . In embodiments in which a warming effect is desired, the compositions should contain higher amounts of warming agents than cooling agent.

It has also been found that topical cooling compositions containing a cooling agent (and smaller amounts or no warming agent) increases the cooling effect of the cooling agent. Therefore, the present invention also provides compositions that include a cooling agent and a silicone-containing component. These compositions contain a majority of a cooling agent, based on the total amount of sensates added to the composition.

Examples of cooling agents that may be included in compositions of the present invention include compounds represented by the formula: 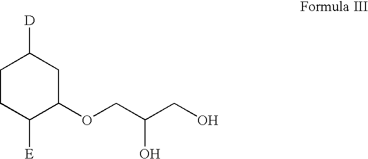
or dermatologically acceptable salts thereof, wherein D is a straight chained or branched, unsubstituted C₁-C₄ alkyl or alkenyl group and E is a straight chained or branched, hydroxy-substituted or unsubstituted C₁-C₄ alkyl group.

In a preferred embodiment, the cooling sensate is represented by the formula: 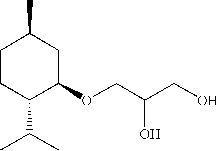
or dermatologically acceptable salts thereof. This compound (3-(1)-menthoxypropane-1, 2-diol) is commercially available from Takasago, Inc. under the name Coolact® 10, and is disclosed in U.S. Pat. No. 4,459,425 which is hereby incorporated by reference.

Other cooling sensates which may be included in compositions of the present invention include, but are not limited to, menthol, menthone, camphor, pulegol, isopulegol, cineol, mint oil, peppermint oil, spearmint oil, eucalyptus oil, 3-1-menthoxypropane-1,2-diol, N-alkyl-p-menthane-3-carboxamide, 3-1-menthoxy-2-methylpropane-1,2-diol, p-menthane-3,8-diol, 2-1-menthoxyethane-1-ol, 3-1-menthoxypropane-1 -ol, 4-1-menthoxybutane-1-ol, 1-(2-hydroxy-4-ethylcyclohexyl)-ethanone, menthyl 3-hydroxybutanoate, menthyl lactate, menthone glycerin ketal, 2-(2-1-menthyloxyethyl)ethanol, menthyl glyoxylate, N-methyl-2,2-isopropylmethyl-3-methylbutanamide, menthyl 2-pyrrolidone-5-carboxylate, monomenthyl succinate, alkali metal salts of monomenthyl succinate, and alkali earth metal salts of monomenthyl succinate, monomenthyl glutarate, alkali metal salts of monomenthyl glutarate, alkali earth metal salts of monomenthyl glutarate, N-[[5-methyl-2-(1-methylethyl) cyclohexyl]carbonyl]glycine, p-menthane-3-carboxylic acid glycerol ester, Menthol propylene glycol carbonate; Menthol ethylene glycol carbonate, and 6-isopropyl-3,9-dimethyl-1,4-dioxaspiro[4.5]decan-2-one.

Other cooling senates are disclosed in U.S. Pat. Nos. 7,030,273 and 6,780,443, which are hereby incorporated by reference in their entirety.

In other embodiments of the present invention, sensates besides warming agents and cooling agents are added to a silicone-containing component in order to enhance the sensate material. For example, in one embodiment of the present invention a tingling sensate is added to a silicone-containing component to enhance the tingling effect of the sensate material.

Silicone-Containing Component

Compositions of the present invention include a silicone-containing component. Although the silicone-containing component, per se, does not generally yield a warming or cooling effect when topically applied, it increases the warming effect of known warming agents. Similarly, the silicone-containing component increases the cooling effect of known cooling agents.

Examples of silicone-containing components that may be used in compositions of the present invention include, but are not limited to, polymers based on methyl silicones, such as cyclomethicone and dimethicone; siloxanes, such as cyclopentasiloxane. Organosilicone emulsifiers including cetyl dimethicone copolyol-polyglyceryl4-isostearate-hexylaurate (ABIL® WE 09) available from Goldschmidt Chemical Corporation, Cetyl Dimethicone Copolyol (ABIL® EM 90), (ABIL® EM 97), Laurylmethicone Copolyol (5200), Cyclomethicone and Dimethicone Copolyol (DC 5225 C and DC 3225 available from Dow Coming and, Cyclopentasiloxane & Dimethicone Copolyol (GE SF 1528) may also be used as a silicone-containing component of the present invention.

The silicone containing component (e.g. polyether siloxane copolymer network compositions) may be utilized as prepared or as the silicone component in emulsions. As is generally known, emulsions comprise at least two immiscible phases one of which is continuous and the other which is discontinuous. Further emulsions may be liquids with varying viscosities or solids. In one embodiment, greater than 50% of the oil phase of the emulsion is silicone, or a compound containing silicone.

Additionally the particle size of the emulsions may be render them microemulsions and when sufficiently small microemulsions may be transparent. For example, the silicone compositions disclosed in U.S. Published Patent Application No. 20060079633, which is hereby incorporated by reference, may be used as prepared or in such an emulsion.

As disclosed in U.S. Published Patent Application No. 20060045890, the silicone-containing component may include silicone resins. The silicone-containing component can be any organosilicone polymer wherein the pure resin is a solid at 25° C. and has a molecular weight, for example, from about 1,000 to about 10,000 daltons. The silicone resin is comprised of siloxane monomer units which may be monoflinctional (“M”) units having the formula [R₃SiO_{0.5}]ₓ, difunctional (“D”) units having the formula [R₂SiO]_{Y}, trifunctional (“T”) units having the formula [RSiO_{1.5}]_{z} and tetrafunctional (“Q”) units having the formula [SiO₂]_{W} where R is preferably methyl but may also be, but is not limited to, C₂ to C₁₆ alkyls, vinyl, phenyl, amine or hydroxyl group. X, Y, Z and W can be any integer such that the molecular weight of the polymer is from about 1,000 to about 10,000 daltons.

The silicone-containing component of the present invention may be comprised of high molecular weight polydimethylsiloxanes (12,500 cSt to gum-type material), such as those described in EP 966972 A1, WO 01/19190 A1, and WO 200122923, the disclosures of which are incorporated herein by reference for their teaching of high molecular weight polydimethylsiloxanes.

The silicone matrix may be comprised of loosely or lightly cross-linked silicone elastomers, for example, Dow Corning® 9040 SILICONE ELASTOMER BLEND (available from Dow Coming Corporation, Midland, Mich.). Loosely or lightly cross-linked silicone elastomers are described in the following U.S. patents which describe loosely cross-linked polydimethylsiloxanes disposed in a volatile silicone solvent (D5), the disclosures of which are hereby incorporated herein by reference: U.S. Pat. Nos. 6,200,581, 6,238,657, 6,177,071, 6,168,782, and 6,207,717. As the volatile silicone solvent evaporates, the lightly or loosely cross-linked silicone elastomer thickens from a paste-like consistency to an elastomeric silicone gel.

Compositions of Warming Agents and Silicone-Containing Compounds

The use level of the warming agent (e.g. vanillyl butyl ether) may vary higher or lower depending on the specific product. In one embodiment of the present invention, the amount of warming agent ranges from about 0.005% to about 5.0%, or from about 0.01% to about 1.0 %. The total amount of warming agent may be decreased by combining a warming agent with a smaller amount of cooling agent. This combination can reduce the likelihood of irritation to the user of the topical composition.

In one embodiment, the amount of silicone-containing components is at least 10% by weight, preferably at least 15% by weight, more preferably at least 20% by weight of the total composition. As shown in the examples, compositions of the present invention may include a combination of one or more sensates of the same type. For example, the compositions may contain a mixture of warming sensates. The selection of the sensate combinations may be selected and adjusted to fine-tune the intensity and duration of the sensate.

Embodiments of the present invention include a cream or ointment base as a vehicle for topical delivery. This is particularly true where the composition is used on dry or peeling skin and when a moisturizing vehicle may otherwise be desirable. Suitable bases include lanolin, SILVADENE™ (silver sulfadiazine) (Hoechst Marion Roussel, Kansas City, Mo.), particularly for treatment of burns, AQUAPHOR™ (Duke Laboratories, South Norwalk, Conn.), and similar bases.

Viscosity building agents may also serve as vehicles for topical delivery, and can be added to aqueous or oil based solutions to form a cream or gel. Examples of viscosity building agents include, but are not limited to, polyethylene glycol, gelatin, chitosan and its derivatives, hydrophilic cellulose (preferably a hydroxyalkylcellulose and more preferably, hydroxymethylcellulose, hydroxyethylcellulose, hydroxypropylcellulose or the like or a mixture thereof), and polyacrylate-polyacrylic acid polymers (e.g., Carbomers and the like).

Other vehicles for topical delivery include, but are not limited to, urea-based emollients, petroleum-based ointments, paste, lotion, liquid paraffin, lanolin, beeswax, vegetable oil, glycerin monostearate, higher alcohols, oil-in-water emulsion, or a water-in-oil emulsion. Further examples of vehicles for topical delivery include those topical agents disclosed in U.S. Pat. Nos. 7,018,660 and 6,994,863, both of which are hereby incorporated by reference in their entirety.

There are many uses for the warming and cooling compositions of the present invention. For example, the compositions of the present invention can be used as a sports rub or gel, as a personal care product (e.g. a soap, shaving foam or shaving gel, a bath soap, a bubble bath, a shower gel, deodorant or antiperspirant). The compositions of the present invention may also be used as a cosmetic product, such as a lipstick, cologne, after shave lotion, or foundation.

Compositions of the present invention may be used as part of a topical medicine or topical analgesic lotion, cream or spray. In such embodiments, compositions of the present invention may be added to an effective amount of a biologically active agent for the treatment or prevention of the desired disease or for achieving the desired physiological effect for which it is administered. Specific indications for active agents can be found in the The Physicians' Desk Reference (58^{th} Ed., 2004, Medical Economics Company, Inc., Montvale, N.J.), and Fauci, AS, et. al., Harrison's Principles of Internal Medicine (14^{th} Ed., 1998, McGraw-Hill Health Professions Division, New York. Both of these references are herein incorporated by reference in their entirety.

Compositions of the present invention may be part of a kit. The kit contains a container for the compositions and packaging that instructs the user to use the composition for its intended purpose. For example, a kit for a sports rub or gel may include, for example, a topical warming composition, a tube that holds the warming composition and instructions (either on the tube or packaging that holds the tube) to the user to topically apply the warming composition to sore muscles. Likewise, a kit for a deodorant composition may include, for example, a topical cooling composition, a tube that holds the topical cooling composition and instructions to the user to topically apply the cooling composition to the underarms as needed.

### EXAMPLES

The following examples illustrate the invention without limitation.

### Example 1

The formulation described below was prepared according to the sequence set forth below Table 1.

TABLE 1
Topical Warming Composition

| | WEIGHT | | |
|---|---|---|---|
| SEQUENCE | PERCENT | INGREDIENT | INCI NAME |
| 1 | 10.00 | DC 3225C | Cyclomethicone |
| | | Formulation Aid | (and) |
| | | (available from Dow | PEG/PPG-18/18 |
| | | Corning, Midland, MI) | Dimethicone |
| 1 | 8.50 | DC 345 Fluid | Cyclopentasiloxane |
| | | (available from Dow | |
| | | Corning, Midland, MI) | |
| 1 | 7.50 | DC 1411 Fluid | Cyclopentasiloxane |
| | | (available from Dow | (and Dimethicone |
| | | Corning, Midland, MI) | |
| 1 | 0.50 | Hotact VBE (available | Vanillyl Butyl Ether |
| | | from Takasago Inc., | |
| | | Rockleigh, NJ) | |
| 2 | 59.20 | Deionized Water | Water |
| 2 | 13.00 | Glycerin (available | Glycerin |
| | | from Ruger Chemical | |
| | | Co., Inc., Linden, NJ) | |
| 2 | 0.20 | Liposorb 0-20 VBE | Polysorbate 80 |
| | | (available from Lipo | |
| | | Chemicals, Inc., | |
| | | Paterson, NJ) | |
| 2 | 1.00 | Sodium Chloride | Sodium Chloride |
| | | (available from Ruger | |
| | | Chemical Co., Inc., | |
| | | Linden, NJ) | |
| 2 | 0.10 | Dowicil 200 (available | Quaternium-15 |
| | | from Dow Chemicals, | |
| | | Inc., Midland, MI) | |

1. Mix Sequence #1 at room temperature.
2. Combine Sequence #2 ingredients and mix until clear and uniform.
3. Add Sequence #2 to Sequence #1 with high speed mixing. Increase mixing speed as emulsion begins to thicken.

To analyze the effect of decreasing the amounts of vanillyl butyl ether, formulations with 0.4 wt%, 0.35 wt%, 0.30 wt% and 0.25 wt% vanillyl butyl ether were prepared by the same procedure as described in Table 1. A panel having 8 members was asked to simultaneously evaluate the topical warming intensity and perceived irritation of these formulations in a blind study over a period of 30 minutes, and the total duration of the warming effect based on application of a 0.5 gram sample on their inside forearm. The panelist were asked to evenly disperse the sample by gently rubbing with their fingers for 10 seconds. These results are set forth below in Table 2.

TABLE 2
Warming Intensity for Topical warming composition of Table 1 having
varying amounts of vanillyl butyl ether (1 = lowest, 6 = highest)

| | 0.50% | 0.40% | 0.35% | 0.30% | 0.25% |
|---|---|---|---|---|---|
| Time | Hotact ® | Hotact ® | Hotact ® | Hotact ® | Hotact ® |
| Interval | VBE | VBE | VBE | VBE | VBE |
| 3 minutes | 1.9 | 2.0 | 1.6 | 1.4 | 1.5 |
| 5 minutes | 3.3 | 2.3 | 2.2 | 1.8 | 1.8 |
| 10 minutes | 4.3 | 2.9 | 2.9 | 2.5 | 2.3 |
| 20 minutes | 4.6 | 2.6 | 3.0 | 2.8 | 2.8 |
| 30 minutes | 4.4 | 2.9 | 2.9 | 2.5 | 2.6 |
| Average | 3.7 | 2.5 | 2.5 | 2.2 | 2.2 |
| over | | | | | |
| 30 minute | | | | | |
| test | | | | | |
| Average | 30 | 30 | 30 | 31 | 29 |
| duration of | | | | | |
| warming | | | | | |
| effect | | | | | |
| beyond 30 | | | | | |
| minute test | | | | | |
| (in minutes) | | | | | |

These formulations were also evaluated in the same manner on the basis of perceived irritation intensity. The results are set forth below in Table 3:

TABLE 3
Perceived Irritation Intensity for topical warming composition of Table 1
having varying amounts of vanillyl butyl ether (1 = lowest, 6 = highest)

| | 0.50% | 0.40% | 0.35% | 0.30% | 0.25% |
|---|---|---|---|---|---|
| Time | Hotact ® | Hotact ® | Hotact ® | Hotact ® | Hotact ® |
| Interval | VBE | VBE | VBE | VBE | VBE |
| 3 minutes | 1.6 | 1.1 | 1.4 | 1.1 | 1.0 |
| 5 minutes | 2.6 | 1.3 | 1.5 | 1.3 | 1.1 |
| 10 minutes | 2.5 | 1.6 | 1.8 | 1.4 | 1.4 |
| 20 minutes | 2.9 | 1.9 | 1.7 | 1.7 | 1.7 |
| 30 minutes | 2.6 | 1.6 | 1.7 | 1.7 | 1.4 |
| Average | 2.4 | 1.5 | 1.6 | 1.4 | 1.3 |
| over 30 | | | | | |
| minute test | | | | | |

These formulations were also evaluated in the same manner on the basis of overall liking and purchasing intent. The results are set forth below in Table 4 and Table 5:

TABLE 4
Overall liking of topical warming composition of Table 1 having
varying amounts of vanillyl butyl ether (1 = lowest, 6 = highest)

| | 0.50% | 0.40% | 0.35% | 0.30% | 0.25% |
|---|---|---|---|---|---|
| | Hotact ® | Hotact ® | Hotact ® | Hotact ® | Hotact ® |
| | VBE | VBE | VBE | VBE | VBE |
| Overall | 2.5 | 2.5 | 3.3 | 3.0 | 3.8 |
| Liking | | | | | |

TABLE 5
Purchase intent of topical warming composition of Table 1 having
varying amounts of vanillyl butyl ether (1 = lowest, 6 = highest)

| | 0.50% | 0.40% | 0.35% | 0.30% | 0.25% |
|---|---|---|---|---|---|
| | Hotact ® | Hotact ® | Hotact ® | Hotact ® | Hotact ® |
| | VBE | VBE | VBE | VBE | VBE |
| Purchase | 2.0 | 2.5 | 2.9 | 2.9 | 3.1 |
| Intent | | | | | |

### Example 2

A warming sports rub formulation was prepared according to the sequence set forth below Table 6 to provide a light, silky cream-gel emulsion with no greasy after-feel:

TABLE 6
Warming sports rub formulation

| | WEIGHT | | |
|---|---|---|---|
| SEQUENCE | PERCENT | INGREDIENT | INCI NAME |
| 1 | 3.00 | Sepigel 305 (available from Dow | Polyacrylamide, C₁₃₋₁₄ |
| | | Corning, Inc., Midland MI) | Isoparaffin and Laureth-7 |
| 1 | 7.00 | DC 9040 Silicone Elastomer Blend | Cyclomethicone and |
| | | (available from Dow Corning, Inc., | Dimethicone Crosspolymer |
| | | Midland MI) | |
| 2 | 75.50 | Deionized Water | Water |
| 2 | 0.50 | LiposerveTM IU (available from | Imidazolindinyl Urea |
| | | Lipo Chemicals, Inc., Paterson, NJ) | |
| 2 | 2.00 | Phytelene ® Colorless Aloe EG 543 | Propylene Glycol, Water, |
| | | (available from Lipo Chemicals, | and Aloe Barbadensis Leaf |
| | | Inc., Paterson, NJ) | Extract |
| 2 | 1.00 | Phytelene ® White Nettle EG 157 | Propylene Glycol, water, |
| | | (available from Lipo Chemicals, | and Lamium Album |
| | | Inc., Paterson, NJ) | (White Nettle) Flower |
| | | | Extract |
| 2 | 0.50 | Liposorb ® L-20 (available from | Polysorbate 20 |
| | | Lipo Chemicals, Inc., Paterson, NJ) | |
| 2 | 0.50 | Hotact VBE (available from | Vanillyl Butyl Ether |
| | | Takasago Inc., Rockleigh, NJ) | |
| 3 | 3.00 | Silkflo ® 366 (available from Lipo | Hydrogenated Polydecene |
| | | Chemicals, Inc., Paterson, NJ) | |
| 3 | 7.00 | Liponate ® NPGC-2 (available from | Neopentyl Glycol |
| | | Lipo Chemicals, Inc., Paterson, NJ) | Dicaprylate/Dicaprate |

PROCEDURE:
1. Mix Sequence #1 together well, at room temperature. A thick, uniform, viscous gel forms.
2. Combine Sequence #2 ingredients and mix until uniform.
3. Slowly add Sequence #2 to Sequence #1 with constant mixing. Product will gel to a cream.
4. Combine Sequence #3 and warm to 40° C. Slowly add to the batch with constant mixing.
Product will re-thicken into a cream gel.

To analyze the effect of decreasing the amounts of vanillyl butyl ether, formulations with 0.4 wt%, 0.35 wt%, 0.30 wt% and 0.25 wt% vanillyl butyl ether were prepared by the same procedure as described in Table 6. A panel having 8 members was asked to simultaneously evaluate the topical warming intensity and perceived irritation of these formulations over a period of 30 minutes (1=lowest, 6=highest), and the total duration of the warming effect based on application of a 0.5 gram sample on their inside forearm as in Example 1. These results are set forth below in Table 7.

TABLE 7
Warming Intensity for sports rub composition of Table 6 having
varying amounts of vanillyl butyl ether (1 = lowest, 6 = highest)

| | 0.50% | 0.40% | 0.35% | 0.30% | 0.25% |
|---|---|---|---|---|---|
| Time | Hotact ® | Hotact ® | Hotact ® | Hotact ® | Hotact ® |
| Interval | VBE | VBE | VBE | VBE | VBE |
| 3 minutes | 1.6 | 1.6 | 1.4 | 1.4 | 1.4 |
| 5 minutes | 2.3 | 1.9 | 1.9 | 1.9 | 1.7 |
| 10 minutes | 2.9 | 2.0 | 2.0 | 2.2 | 1.8 |
| 20 minutes | 3.0 | 1.8 | 1.7 | 1.9 | 1.9 |
| 30 minutes | 3.0 | 1.6 | 1.5 | 1.5 | 1.5 |
| Average | 2.6 | 1.8 | 1.7 | 1.8 | 1.7 |
| over | | | | | |
| 30 minute | | | | | |
| test | | | | | |
| Average | 32 | 12 | 10 | 11 | 14 |
| duration of | | | | | |
| warming | | | | | |
| effect | | | | | |
| beyond 30 | | | | | |
| minute test | | | | | |
| (in minutes) | | | | | |

These formulations were also evaluated in the same manner on the basis of perceived irritation intensity. The results are set forth below in Table 8:

TABLE 8
Perceived Irritation Intensity for topical warming composition of Table 6
having varying amounts of vanillyl butyl ether (1 = lowest, 6 = highest)

| | 0.50% | 0.40% | 0.35% | 0.30% | 0.25% |
|---|---|---|---|---|---|
| Time | Hotact ® | Hotact ® | Hotact ® | Hotact ® | Hotact ® |
| Interval | VBE | VBE | VBE | VBE | VBE |
| 3 minutes | 1.1 | 1.1 | 1.1 | 1.2 | 1.0 |
| 5 minutes | 1.5 | 1.1 | 1.1 | 1.1 | 1.1 |
| 10 minutes | 1.8 | 1.2 | 1.2 | 1.2 | 1.2 |
| 20 minutes | 1.9 | 1.2 | 1.1 | 1.1 | 1.1 |
| 30 minutes | 1.9 | 1.1 | 1.1 | 1.1 | 1.0 |
| Average | 1.6 | 1.1 | 1.1 | 1.1 | 1.1 |
| over 30 | | | | | |
| minute test | | | | | |

These formulations were also evaluated in the same manner on the basis of overall liking and purchasing intent. The results are set forth below in Table 9 and Table 10:

TABLE 9
Overall liking sports rub composition of Table 6 having varying
amounts of vanillyl butyl ether (1 = lowest, 6 = highest)

| | 0.50% | 0.40% | 0.35% | 0.30% | 0.25% |
|---|---|---|---|---|---|
| | Hotact ® | Hotact ® | Hotact ® | Hotact ® | Hotact ® |
| | VBE | VBE | VBE | VBE | VBE |
| Overall | 2.9 | 1.7 | 2.0 | 2.3 | 2.3 |
| Liking | | | | | |

TABLE 10
Purchase intent of sports rub composition of Table 6 having
varying amounts of vanillyl butyl ether (1 = lowest, 6 = highest)

| | | Formula | Formula II | Formula II | Formula II |
|---|---|---|---|---|---|
| | Formula II | II with | with | with | with |
| | with 0.50% | 0.40% | 0.35% | 0.30% | 0.25% |
| | Hotact ® | Hotact ® | Hotact ® | Hotact ® | Hotact ® |
| | VBE | VBE | VBE | VBE | VBE |
| Purchase | 2.6 | 1.7 | 1.9 | 1.9 | 2.2 |
| Intent | | | | | |

### Example 3

The warming sports rub formulation of Example 2, Table 6 was reproduced, except that 0.50% vanillyl butyl ether was replaced with: (a) 0.50% vanillyl ethyl ether (b) 0.50% vanillin-1,2-hexylene glycol acetal; (c) 0.50% vanillin-1-butoxyglycerol acetal (d) 0.25% vanillyl butyl ether plus 0.25% vanillyl ethyl ether; and (e) 0.40% vanillyl butyl ether and 0. 10% 3-(1)-menthoxypropane-1,2-diol to provide five new formulations. Perceived irritation intensity, overall liking, and purchase intent was evaluated as in Example 2, and the results are set forth below:

TABLE 11
Warming Intensity for sports rub composition of Table 6 which different warming
sensates replacing vanillyl butyl ether (1 = lowest, 6 = highest)

| | | | | 0.25% | |
|---|---|---|---|---|---|
| | | | | vanillyl | |
| | | | | butyl | |
| | | 0.50% | | ether | |
| | | vanillin- | 0.50% | plus | |
| | 0.50% | 1,2-- | vanillin- | 0.25% | |
| | vanillyl | hexylene | 1- | vanillyl | 0.40% vanillyl butyl |
| | ethyl | glycol | butoxyglycerol | ethyl | ether and 0.10% 3-(1)- |
| | ether | acetal | acetal | ether | menthoxypropane-1,2-diol |
| 3 min. | 1.3 | 1.8 | 1.7 | 1.5 | 1.8 |
| 5 min. | 1.3 | 1.9 | 1.9 | 1.6 | 2.3 |
| 10 min. | 1.5 | 2.0 | 1.8 | 2.0 | 2.8 |
| 20 min. | 1.3 | 1.8 | 2.0 | 1.8 | 3.3 |
| 30 min. | 1.3 | 1.3 | 1.4 | 1.6 | 3.0 |
| Ave. over | 1.3 | 1.8 | 1.8 | 1.7 | 2.6 |
| 30 min. | | | | | |
| test | | | | | |
| Average | 1 | 2 | 6 | 9 | 35 |
| duration | | | | | |
| of | | | | | |
| warming | | | | | |
| effect | | | | | |
| beyond | | | | | |
| 30 minute | | | | | |
| test (in | | | | | |
| minutes) | | | | | |
| effect | | | | | |

TABLE 12
Perceived Irritation Intensity for sports rub composition of Table 6 which different
warming sensates replacing vanillyl butyl ether (1 = lowest, 6 = highest)

| | | | | 0.25% | |
|---|---|---|---|---|---|
| | | | | vanillyl | |
| | | | | butyl | |
| | | 0.50% | | ether | |
| | | vanillin- | | plus | |
| | 0.50% | 1,2-- | 0.50% | 0.25% | 0.40% |
| | vanillyl | hexylene | vanillin-1- | vanillyl | vanillyl butyl ether |
| Time | ethyl | glycol | butoxyglycerol | ethyl | and 0.10% 3-(1)- |
| Interval | ether | acetal | acetal | ether | menthoxypropane-1,2-diol |
| 3 min. | 1.0 | 1.1 | 1.2 | 1.1 | 1.0 |
| 5 min. | 1.0 | 1.4 | 1.4 | 1.0 | 1.3 |
| 10 min. | 1.0 | 1.3 | 1.4 | 1.1 | 1.8 |
| 20 min. | 1.0 | 1.2 | 1.2 | 1.0 | 1.8 |
| 30 min. | 1.0 | 1.1 | 1.1 | 1.0 | 1.9 |
| Average | 1.0 | 1.2 | 1.3 | 1.0 | 1.6 |
| over 30 min. | | | | | |
| test | | | | | |

TABLE 13
Overall liking of sports rub composition of Table 6 which different
warming sensates replacing vanillyl butyl ether (1 = lowest, 6 = highest)

| | | | | 0.25% | |
|---|---|---|---|---|---|
| | | | | vanillyl | 0.40% |
| | | | | butyl | vanillyl |
| | | 0.50% | | ether | butyl ether |
| | | vanillin- | | plus | and 0.10% |
| | 0.50% | 1,2-- | 0.50% | 0.25% | 3-(1)- |
| | vanillyl | hexylene | vanillin-1- | vanillyl | menthoxy- |
| | ethyl | glycol | butoxyglycerol | ethyl | propane-1, |
| | ether | acetal | acetal | ether | 2-diol |
| Overall | 1.1 | 1.9 | 2.0 | 1.9 | 3.0 |
| Liking | | | | | |

TABLE 14
Purchase intent for sports rub composition of Table 6 which different
warming sensates replacing vanillyl butyl ether (1 = lowest, 6 = highest)

| | | | | 0.25% | 0.40% |
|---|---|---|---|---|---|
| | | | | vanillyl | vanillyl |
| | | | | butyl | butyl |
| | | 0.50% | | ether | ether and |
| | | vanillin- | | plus | 0.10% 3-(1)- |
| | 0.50% | 1,2-- | 0.50% | 0.25% | menthoxy- |
| | vanillyl | hexylene | vanillin-1- | vanillyl | propane-1, |
| | ethyl | glycol | butoxyglycerol | ethyl | 2- |
| | ether | acetal | acetal | ether | diol |
| Purchase | 1.1 | 1.8 | 1.7 | 1.8 | 2.4 |
| Intent | | | | | |

### Example 4

The topical warming composition of Example 1, Table 1 was reproduced, except that 0.50% vanillyl butyl ether was replaced with: (a) 0.25% vanillyl butyl ether and (b) 0.25% vanillyl butyl ether plus 0.4% of a proprietary fragrance composition manufactured by Takasago, Inc. The topical sports rub of Example 2, Table 6 was also reproduced, except that 0.50% vanillyl butyl ether was replaced with: (c) 0.25% vanillyl butyl ether and (d) 0.25% vanillyl butyl ether plus 0.4% of the same proprietary fragrance composition manufactured by Takasago, Inc.

TABLE 15
Warming Intensity for compositions of Table 1 and Table 6 with reduced
amounts of vanillyl butyl ether with and without fragrance (1 = lowest,
6 = highest)

| | | | | 0.25% |
|---|---|---|---|---|
| | | 0.25% | | vanillyl |
| | | vanillyl butyl | | butyl |
| | | ether plus | | ether plus |
| | 0.25% | 0.4% | | 0.4% |
| | vanillyl | proprietary | 0.25% vanillyl | proprietary |
| | butyl ether | fragrance | butyl ether | fragrance |
| | replaced in | replaced in | replaced in | Formulation |
| | Formulation | Formulation | Formulation of | of |
| | of Table 1 | of Table 1 | Table 6 | Table 6 |
| 3 min. | 1.5 | 1.5 | 1.4 | 1.6 |
| 5 min. | 1.8 | 1.9 | 1.7 | 1.6 |
| 10 min. | 2.3 | 3.1 | 1.8 | 2.0 |
| 20 min. | 2.8 | 3.5 | 1.9 | 1.5 |
| 30 min. | 2.6 | 2.9 | 1.5 | 1.4 |
| Ave. | 2.2 | 2.6 | 1.7 | 1.6 |
| over 30 min. | | | | |
| test | | | | |
| Average | 29 | 28 | 14 | 11 |
| duration | | | | |
| of | | | | |
| warming | | | | |
| effect | | | | |
| beyond | | | | |
| 30 | | | | |
| minute | | | | |
| test (in | | | | |
| minutes) | | | | |

TABLE 16
Perceived Irritation Intensity of compositions of Table 1 and 6 which different
warming sensates replacing vanillyl butyl ether (1 = lowest, 6 = highest)

| | | 0.25% vanillyl | | 0.25% vanillyl |
|---|---|---|---|---|
| | | ethyl ether plus | | ethyl ether plus |
| | 0.25% vanillyl | 0.4% proprietary | 0.25% vanillyl | 0.4% proprietary |
| | ethyl ether | fragrance | ethyl ether | fragrance |
| | replaced in | replaced in | replaced in | replaced in |
| | Formulation of | Formulation of | Formulation of | Formulation of |
| Time Interval | Table 1 | Table 1 | Table 6 | Table 6 |
| 3 min. | 1.0 | 1.3 | 1.0 | 1.4 |
| 5 min. | 1.1 | 1.6 | 1.1 | 1.3 |
| 10 min. | 1.4 | 1.8 | 1.2 | 1.4 |
| 20 min. | 1.7 | 1.8 | 1.1 | 1.3 |
| 30 min. | 1.4 | 1.8 | 1.0 | 1.1 |
| Average | 1.3 | 1.7 | 1.1 | 1.3 |
| over 30 min. | | | | |
| test | | | | |

TABLE 17
Overall liking of compositions of Table 1 and 6 which different warming
sensates replacing vanillyl butyl ether (1 = lowest, 6 = highest)

| | | 0.25% | | |
|---|---|---|---|---|
| | | vanillyl ethyl | | 0.25% vanillyl |
| | 0.25% | ether plus 0.4% | 0.25% | ethyl ether plus |
| | vanillyl | proprietary | vanillyl | 0.4% proprietary |
| | ethyl ether | fragrance | ethyl ether | fragrance |
| | replaced in | replaced in | replaced in | replaced in |
| | Formulation | Formulation of | Formulation | Formulation of |
| | of Table 1 | Table 1 | of Table 6 | Table 6 |
| Overall | 3.8 | 3.3 | 2.3 | 1.9 |
| Liking | | | | |

TABLE 18
Purchase intent for compositions of Table 1 and 6 which different
warming sensates replacing vanillyl butyl ether (1 = lowest, 6 = highest)

| | | 0.25% | | 0.25% |
|---|---|---|---|---|
| | | vanillyl ethyl | | vanillyl ethyl |
| | 0.25% | ether plus 0.4% | 0.25% | ether plus 0.4% |
| | vanillyl | proprietary | vanillyl | proprietary |
| | ethyl ether | fragrance | ethyl ether | fragrance |
| | replaced in | replaced in | replaced in | replaced in |
| | Formulation | Formulation of | Formulation | Formulation of |
| | of Table 1 | Table 1 | of Table 6 | Table 6 |
| Purchase | 3.1 | 2.9 | 2.2 | 2.0 |
| Intent | | | | |

### Example 5

The sports rub formulation of Example 2, Table 6 was reproduced, except that 0.50% vanillyl butyl ether was replaced with: (a) 0. 10% vanillyl butyl ether and 0.40% 3-(1)-menthoxypropane-1, 2-diol; and (b) 1.0% 3-(l)-menthoxypropane-1, 2-diol to provide two new formulations. In contrast to the warming agent that is included in Example 2, the sports rub prepared in this example contains a majority cooling agent (based on total amount of sensate in composition) and imparts a cooling sensation to the user. Cooling intensity, perceived irritation intensity, overall liking, and purchase intent was evaluated as in Example 2, and the results are set forth below:

TABLE 19
Cooling Intensity for sports rub composition of Table 6 which different
sensates replacing vanillyl butyl ether (1 = lowest, 6 = highest)

| | 0.10% | |
|---|---|---|
| | vanillyl | |
| | butyl | |
| | ether and | |
| | 0.40% 3- | |
| | (1)- | 1.0% 3-(1)- |
| | menthoxypropane- | menthoxypropane-1,2- |
| | 1,2-diol | diol |
| 3 min. | 1.7 | 1.5 |
| 5 min. | 1.7 | 1.5 |
| 10 min. | 1.4 | 1.7 |
| 20 min. | 1.2 | 1.8 |
| 30 min. | 1.0 | 1.5 |
| Ave. | 1.4 | 1.6 |
| over 30 min. | | |
| test | | |
| Average | 0 | 3 |
| duration | | |
| of | | |
| warming | | |
| effect | | |
| beyond | | |
| 30 | | |
| minute | | |
| test (in | | |
| minutes) | | |
| effect | | |

TABLE 20
Perceived Irritation Intensity for sports rub composition
of Table 6 which different warming sensates replacing
vanillyl butyl ether (1 = lowest, 6 = highest)

| | 0.10% vanillyl | 1.0% 3- |
|---|---|---|
| | butyl ether and | (1)- |
| | 0.40% 3-(1)- | menthoxypropane- |
| Time | menthoxypropane- | 1, |
| Interval | 1,2-diol | 2-diol |
| 3 min. | 1.1 | 1.0 |
| 5 min. | 1.3 | 1.0 |
| 10 min. | 1.0 | 1.0 |
| 20 min. | 1.0 | 1.0 |
| 30 min. | 1.0 | 1.2 |
| Average | 1.1 | 1.0 |
| over 30 min. | | |
| test | | |

TABLE 21
Overall liking of sports rub composition of Table 6 which different
warming sensates replacing vanillyl butyl ether (1 = lowest, 6 = highest)

| | 0.10% vanillyl | 1.0% 3- |
|---|---|---|
| | butyl ether and | (1)- |
| | 0.40% 3-(1)- | menthoxypropane- |
| | menthoxypropane- | 1, |
| | 1,2-diol | 2-diol |
| Overall | 1.3 | 1.3 |
| Liking | | |

TABLE 22
Purchase intent for sports rub composition of Table 6 which different
warming sensates replacing vanillyl butyl ether (1 = lowest, 6 = highest)

| | 0.10% | |
|---|---|---|
| | vanillyl | |
| | butyl | |
| | ether and | |
| | 0.40% 3- | 1.0% 3- |
| | (1)- | (1)- |
| | menthoxypropane- | menthoxypropane- |
| | 1,2-diol | 1,2-diol |
| Purchase | 1.2 | 1.2 |
| Intent | | |

### Comparative Example 1

The formulation described below was prepared according to the sequence set forth below Table 19.

TABLE 23
Hydrocarbon Emulsion Lotion

| Sequence | Ingredient | INCI Name | Weight % |
|---|---|---|---|
| 1 | DI water | Water | 59.65 |
| 1 | Liponic EG-1 | Glycereth-26 | 6.00 |
| 1 | Liposerve PP | Phenoxyethanol and | 1.00 |
| | | Methylparaben and | |
| | | Ethylparaben and | |
| | | Butylparaben and | |
| | | Propylparaben and | |
| | | Isobutylparaben | |
| 1 | Carbopol 980 (2%) | Carbomer | 12.50 |
| 1 | Na₂X | Disodium EDTA | 0.05 |
| 2 | Lipomulse 165 | Glyceryl Stearate | 6.00 |
| | | and PEG-100 | |
| | | Stearate | |
| 2 | Lipowax P | Cetearyl Alcohol | 0.75 |
| | | and Polysorbate 60 | |
| 2 | Silkflo 362 | Hydrogenated | 8.00 |
| | | Didecene | |
| 2 | Pnalane L-14E | Hydrogenated | 0.50 |
| | | Polyisobutene | |
| 2A | Hotact VBE | Vanillyl Butyl Ether | 0.50 |
| | NaOH (18%) | Sodium Hydroxidce | 0.25 |
| | DI water | Water | 1.00 |
| | Liposerve IU | Imidazolidinyl Urea | 0.30 |

Sequence
1. Heat water phase 1 to 80° C. with propeller mixer.
2. Heat phase 2 to 80° C. and mix until clear and uniform.
3. Add phase 2 to phase 1 with propeller mixing.
4. Cool to 50° C. and add sequence 2A to batch.
5. Add sequence 3 to batch and continue cooling.

6. Cool to 30-35° C. and add sequence 4. Cool to room temperature.

TABLE 24
A high foaming cleansing gel

| | WEIGHT | | |
|---|---|---|---|
| SEQUENCE | PERCENT | INGREDIENT | INCI NAME |
| 1 | 46.10 | Deionized Water | Water |
| 1 | 2.00 | Liponic ™ EG-1 | Glycereth-26 |
| 1 | 0.05 | Uvinul MS-40 (37) | Benzophenone-4 |
| 1 | 0.10 | Dissolvine Na4 (92) | Tetrasodium EDTA |
| 1 | 0.20 | Methylparaben | Methylparaben |
| | | (119) | |
| 1 | 0.05 | Citric Acid (119) | Citric Acid |
| 1 | 1.00 | Lipamide ® | Lactamide MEA |
| | | LMEA (1) | |
| 2 | 37.00 | Standapol ES-2 (20) | Sodium Laureth |
| | | | Sulfate |
| 2 | 0.30 | Lipopeg ® | PEG-150 Distearate |
| | | 6000DS (1) | |
| 2 | 3.00 | Comperlan 100 (20) | Cocamide MEA |
| 3 | 5.00 | Velvetex BK-35 | Cocamidopropyl |
| | | (20) | Betaine |
| 5 | 0.05 | Kathon CG (14) | Methyl- |
| | | | chloroisothiazolinone |
| | | | (and) |
| | | | Methylisothiazolinone |
| 6 | 4.00 | Liposorb ® | Polysorbate-20 |
| | | L-20 (1) | |
| 6 | 0.40 | Hotact VBE (125) | Vanillyl Butyl Ether |
| 6 | 0.75 | Fragrance (43) | Fragrance |

(1) Lipo Chemicals
(14) Rohm & Haas Company, Inc.
(20) Cognis Corporation
(37) BASF Corporation
(43) Premier Specialties, Inc.
(92) Akzo Nobel Chemicals, Inc.
(119) Ruger Chemical Co., Inc.
(125) Lipo (Takasago)

PROCEDURE:
1. In main kettle, heat water to 78-80° C. with medium speed propeller mixing. Mix until uniform.
2. Combine Sequence #2 ingredients and heat to 70-75° C. Mix until clear and uniform.
3. Add Sequence #2 to Sequence #1 with medium speed propeller mixing.
4. Add Sequence #3 to batch and mix until completely dissolved.
5. Combine Sequence #4 ingredients and heat to 60° C.
6. Add Sequence #4 to batch with medium speed propeller mixing. Begin cooling.
7. At 42° C., add sequence #5 and premixed Sequence #6 ingredients to batch.
8. At 30° C., add Sequence #7 to batch.
SPECIFICATIONS:
pH: 7.0-7.5
Viscosity: LVT#3 ( 12 rpm-4200 cps ±10%

Stability: 30 days at 50° C.

TABLE 25
Shampoo

| | WEIGHT | | |
|---|---|---|---|
| SEQUENCE | PERCENT | INGREDIENT | INCI NAME |
| 1 | 39.4 | DI water | Water |
| 1 | 0.75 | Liposerve PP | Phenoxyethanol and |
| | | | Methylparaben and |
| | | | Ethylparaben and |
| | | | Butylparaben and |
| | | | Propylparaben and |
| | | | Isobutylparaben |
| 1 | 0.50 | Lipoquat R | Ricinoleamidopropyl |
| | | | Ethyldimonium |
| | | | Ethosulfate |
| 1 | 0.10 | Panthenol | Panthenol |
| 1 | 3.00 | Liponic EG-1 | Glycereth-26 |
| 1 | 1.00 | Lipopeg 6000DS | PEG-150 Distearate |
| 2 | 25.00 | Bio-Terge AS-40 | Sodium C14-16 Olefin |
| | | | Sulfonate |
| 2 | 15.00 | Standapol ES-2 | Sodium Laureth Sulfate |
| 3 | 10.00 | Mackam CET | Cetyl Betaine |
| 3 | 1.00 | Mackamide CMA | Cocamide MEA |
| 4 | 2.00 | Tegosoft GMC-6 | PEG-6 Caprylic/Capric |
| | | | Glycerides |
| 5 | 0.05 | Kathon CG | Methyl- |
| | | | chloroisothiazolinone and |
| | | | Methylisothiazolinone |
| 6 | 2.00 | Butylene glycol | Butylene Glycol |
| 6 | 0.20 | Hotact VBE | Vanillyl Butyl Ether |

1. Heat Sequence 1 to 75-80 IC with propeller mixing.
2. Add Sequence 2 ingredients to Sequence 1 one at a time being careful not to aerate the batch.
3. Heat Sequence 3 to 69-65 IC and add to batch
4. Add Sequence 4 to batch and begin cooling.
5. At 50° C., add sequence 5 and premixed Sequence 6 to batch.

6. Cool to 30-35° C.

TABLE 26
Gel

| | WEIGHT | | |
|---|---|---|---|
| SEQUENCE | PERCENT | INGREDIENT | INCI NAME |
| 1 | 48.15 | DI Water | Water |
| 1 | 5.00 | Liponic EG-1 | Glycereth-26 |
| 2 | 15.00 | Carbopol ETD 2001 | Carbomer |
| 3 | 30.00 | SD Alcohol 40B | SD Alcohol 40-B |
| 4 | 0.60 | TEA 99% | Triethanolamine |
| 4 | 1.0 | DI water | Water |
| 5 | 0.25 | Hotact VBE | Vanillyl Butyl Ether |

1. Combine Sequence 1 ingredients with propeller mixing.
2. Add Sequence 2 and Sequence 3 to Sequence 1. Mix well between additions.
3. Add premixed Sequence 4 to batch. Increase mixing as batch begins to thicken.
4. Add sequence 5 to batch. Mix until uniform.

A topical warming effect was not observed in any of the formulations of this comparative example. These formulations did not contain a silicone-containing component.

The above-mentioned patents, applications, test methods, and publications are hereby incorporated by reference in their entirety.

Many variations of the present invention will suggest themselves to those skilled in the art in light of the above detailed description. All such obvious variations are within the fully intended scope of the appended claims.

1. A warming composition comprising:
(a) at least warming agent; and
(b) at least one silicone-containing component.

2. The warming composition of claim 1, wherein the warming agent is selected from the group consisting of:
(a) a compound represented by the following formula: 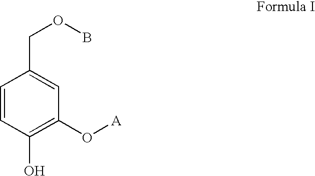
wherein A is a unsubstituted, branched or straight-chained C₁-C₃ alkyl group and B is a hydrogen, or an unsubstituted, branched or straight-chained C₁-C₇ alkyl group, or a dermatologically acceptable salt thereof;
(b) a compound represented by the formula. 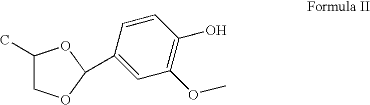
wherein C is an unsubstituted, branched or straight-chained C₂-C8 alkyl group, optionally interrupted by an oxygen atom, or a dermatologically acceptable salt thereof; or
(c) a compound selected from the group consisting of capsaicin, gingerol, vanillyl ethyl ether, vanillyl propyl ether, vanillyl butyl ether, vanillyl pentyl ether, vanillyl hexyl ether, vanillyl butyl ether acetate, 4-(1-menthoxymethyl)-2-phenyl-1,3-dioxolan, 4-(1-menthoxymethyl)-2-(3′,4′-dihydroxyphenyl)-1,3-dioxolan, 4-(1-menthoxymethyl)-2-(2′-hydroxy-3′-methoxyphenyl)-1,3-dioxolan, 4-(1-menthoxymethyl)-2-(4′-methoxyphenyl)-1,3-dioxolan, 4-(1-menthoxymethyl)-2-(3′,4′-methylenedioxyphenyl)-1,3-dioxolan, 4-(1-menthoxymethyl)-2-(3′-methoxy-4′-hydroxyphenyl)-1,3-dioxolan, red pepper oil, red pepper oleoresin, ginger oleoresin, nonylic acid vanillyl amide, jambu oleoresin, Zanthoxylum piperitum extract, sanshool I, sanshool II, sanshoamide, black pepper extract, chavicine, piperine, and spilanthol.

3. The warming composition of claim 1, wherein the warming agent is selected from vanillyl butyl ether and vanillyl ethyl ether.

4. The warming composition of claim 1, wherein the warming agent is vanillyl butyl ether.

5. The warming composition of any one of the preceding claims, wherein the silicone-containing component is selected from cyclomethicone, cyclopentasiloxane, and dimethicone.

6. The warming composition of any one of the preceding claims, wherein the composition contains at least 10% by weight of silicone-containing components, based on the total weight of the composition.

7. The warming composition of any one of the preceding claims, further comprising a cooling agent.

8. The warming composition of any one of the preceding claims, wherein the cooling agent is selected from the group consisting of:
(a) a compound represented by the formula: 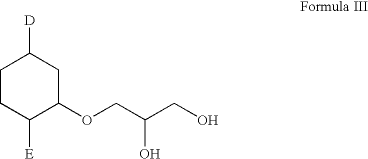
or dermatologically acceptable salts thereof, wherein D is a straight chained or branched, unsubstituted C₁-C₄ alkyl or alkenyl group and E is a straight chained or branched, hydroxy-substituted or unsubstituted C₁-C₄ alkyl or alkenyl group; and
(b) a compound selected from the group consisting of Other cooling sensates which may be included in compositions of the present invention include, but are not limited to, menthol, menthone, camphor, pulegol, isopulegol, cineol, mint oil, peppermint oil, spearmint oil, eucalyptus oil, 3-1-menthoxypropane-1,2-diol, N-alkyl-p-menthane-3-carboxamide, 3-1-menthoxy-2-methylpropane-1,2-diol, p-menthane-3,8-diol, 2-1-menthoxyethane-1-ol, 3-1-menthoxypropane-1-ol, 4-1-menthoxybutane- 1 -ol, 1-(2-hydroxy-4-ethylcyclohexyl)-ethanone, menthyl 3-hydroxybutanoate, menthyl lactate, menthone glycerin ketal, 2-(2-1-menthyloxyethyl)ethanol, menthyl glyoxylate, N-methyl-2,2-isopropylmethyl-3-methylbutanamide, menthyl 2-pyrrolidone-5-carboxylate, monomenthyl succinate, alkali metal salts of monomenthyl succinate, and alkali earth metal salts of monomenthyl succinate, monomenthyl glutarate, alkali metal salts of monomenthyl glutarate, alkali earth metal salts of monomenthyl glutarate, N-[[5-methyl-2-(1-methylethyl) cyclohexyl]carbonyl]glycine, p-menthane-3-carboxylic acid glycerol ester, Menthol propylene glycol carbonate; Menthol ethylene glycol carbonate, and 6-isopropyl-3,9-dimethyl-1,4-dioxaspiro[4.5]decan-2-one.

9. The warming composition of claim 8, wherein the cooling sensate is (3-(1)-menthoxypropane-1,2-diol.

10. A cooling composition comprising:
(a) a cooling agent; and
(b) a silicone-containing component.

11. The cooling composition of claim 10, wherein the cooling agent is selected from the group consisting of:
(a) a compound represented by the formula: 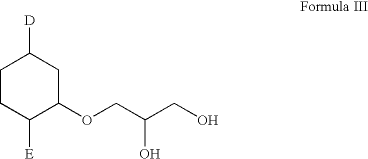
or dermatologically acceptable salts thereof, wherein D is a straight chained or branched, unsubstituted C₁-C₄ alkyl or alkenyl group and E is a straight chained or branched, hydroxy-substituted or unsubstituted C₁-C₄ alkyl or alkenyl group; and
(b) a compound selected from the group consisting of Other cooling sensates which may be included in compositions of the present invention include, but are not limited to, menthol, menthone, camphor, pulegol, isopulegol, cineol, mint oil, peppermint oil, spearmint oil, eucalyptus oil, 3-1-menthoxypropane-1,2-diol, N-alkyl-p-menthane-3-carboxamide, 3-1-menthoxy-2-methylpropane-1,2-diol, p-menthane-3,8-diol, 2-1-menthoxyethane-1-ol, 3-1-menthoxypropane-1-ol, 4-1-menthoxybutane-1-ol, 1-(2-hydroxy-4-ethylcyclohexyl)-ethanone, menthyl 3-hydroxybutanoate, menthyl lactate, menthone glycerin ketal, 2-(2-1-menthyloxyethyl)ethanol, menthyl glyoxylate, N-methyl-2,2-isopropylmethyl-3-methylbutanamide, menthyl 2-pyrrolidone-5-carboxylate, monomenthyl succinate, alkali metal salts of monomenthyl succinate, and alkali earth metal salts of monomenthyl succinate, monomenthyl glutarate, alkali metal salts of monomenthyl glutarate, alkali earth metal salts of monomenthyl glutarate, N-[[5-methyl-2-(1-methylethyl) cyclohexyl]carbonyl]glycine, p-menthane-3-carboxylic acid glycerol ester, Menthol propylene glycol carbonate; Menthol ethylene glycol carbonate, and 6-isopropyl-3,9-dimethyl-1,4-dioxaspiro[4.5]decan-2-one.

12. A kit comprising a sports rub gel or creme, said sports rub gel or creme comprising a composition of any one of the preceding claims.

13. A kit comprising a shaving gel or creme, said shaving gel or creme comprising a composition of any one of claims 1-12.

14. A kit comprising a personal care product, said personal care product comprising a composition of any one of claims 1-12.

15. A method of relieving sore muscles comprising topically applying a composition of any one of claims 1-12.

16. A method of preparing a topical medicine or topical analgesic lotion cream or spray comprising adding the composition of any one of claims 1-12 to a biologically active agent.

17. A method of increasing the effect of a sensate material comprising adding an effective amount of a silicone-containing component to a composition comprising the sensate material.

18. The method of claim 17 wherein the sensate material is a warming agent.

19. The method of claim 17 wherein the sensate material is a cooling agent.

20. The method of claim 17, wherein the sensate material is a tingling agent.

21. The composition of any one of claims 1-12, wherein the composition is in a form suitable for topical delivery.
